(19) United States
(12) Patent Application Publication
(10) Pub. No.: US 2023/0338681 A1
(43) Pub. Date: Oct. 26, 2023

(54) ANESTHESIA MACHINE, VETERINARY ANESTHESIA MACHINE, AND FAN ASSEMBLY

(71) Applicant: Shenzhen Mindray Animal Medical Technology Co., Ltd., Shenzhen (CN)

(72) Inventors: Jicheng LI, Shenzhen (CN); Yiwei CHEN, Shenzhen (CN)

(73) Assignee: Shenzhen Mindray Animal Medical Technology Co., Ltd., Shenzhen (CN)

(21) Appl. No.: 18/345,047

(22) Filed: Jun. 30, 2023

Related U.S. Application Data
(63) Continuation of application No. PCT/CN2020/142436, filed on Dec. 31, 2020.

(51) Int. Cl. A61M 16/01; A61M 16/10; A61M 16/00

(52) U.S. Cl. CPC A61M 16/01; A61M 16/104; A61M 16/0066; A61M 2205/42; A61M 2205/3666

(57) ABSTRACT

The invention provides an anesthesia machine comprising a driving gas branched path, a fresh gas branched path, and a breathing circuit. The fresh gas branched path delivers fresh gas having anesthetic into the breathing circuit. The driving gas branched path drives fresh gas from the breathing circuit to a patient. The driving gas branched path comprises a fan assembly, which comprises a housing, a fan, and a heat-dissipating member. The fan is disposed in a first inner chamber, and drives air into the first inner chamber. The heat-dissipating member conducts to the housing the heat generated by the motor.

### CROSS-REFERENCE

This application is a continuation of International Application No. PCT/CN2020/142436, filed on Dec. 31, 2020, which is incorporated herein by reference in its entirety.

### TECHNICAL FIELD

The disclosure relates to the field of medical instruments, and in particular to an anesthesia machine, a veterinary anesthesia machine, and a fan assembly.

### BACKGROUND

A fan assembly of the existing anesthesia machine uses a heat sink or heat pipe to dissipate heat generated in operation of a motor to the outside, and uses a cooling fan to dissipate the heat forcedly. This heat dissipation structure is complex and costly.

### SUMMARY

In view of this, the disclosure provides an anesthesia machine, a veterinary anesthesia machine, and a fan assembly.

In a first aspect of the disclosure, provided is an anesthesia machine, including a driving gas branch, a fresh gas branch, and a breathing circuit, where the fresh gas branch is configured to deliver fresh gas with anesthetic gas into the breathing circuit, the driving gas branch is configured to push the fresh gas from the breathing circuit to a patient, the driving gas branch includes a fan assembly, the fan assembly includes a housing, a fan and a first heat-dissipating member, the housing includes a first inner cavity, a first air inlet communicating with the first inner cavity, and a first air outlet communicating with the first inner cavity, the fan is disposed in the first inner cavity, the fan is configured to drive air from the first air inlet into the first inner cavity and blow the air out from the first air outlet, and the fan includes:
a volute;
an impeller, rotatably mounted in the volute; and
a motor, mounted in the volute and connected to the impeller, and configured to drive the impeller to rotate;
where the first heat-dissipating member is mounted on the motor and connected to the housing, and the first heat-dissipating member is configured to conduct heat generated by the motor to the housing.

In a second aspect of the disclosure, provided is a veterinary anesthesia machine, including a driving gas branch, a fresh gas branch, and a breathing circuit, where the fresh gas branch is configured to deliver fresh gas with anesthetic gas into the breathing circuit, the driving gas branch is configured to push the fresh gas from the breathing circuit to a target object, the driving gas branch includes a fan assembly, the fan assembly includes a housing, a fan, and a first heat-dissipating member, the housing includes a first inner cavity, a first air inlet communicating with the first inner cavity, and a first air outlet communicating with the first inner cavity, the fan is disposed in the first inner cavity, the fan is configured to drive air from the first air inlet into the first inner cavity and blow the air out from the first air outlet, and the fan includes:
a volute;
an impeller, rotatably mounted in the volute; and
a motor, mounted in the volute and connected to the impeller, and configured to drive the impeller to rotate;
where the first heat-dissipating member is mounted on the motor and connected to the housing, and the first heat-dissipating member is configured to conduct heat generated by the motor to the housing.

In a third aspect of the disclosure, provided is a fan assembly, including a housing, a fan, and a first heat-dissipating member, the housing includes a first inner cavity, a first air inlet communicating with the first inner cavity, and a first air outlet communicating with the first inner cavity, the fan is disposed in the first inner cavity, the fan is configured to drive air from the first air inlet into the first inner cavity and blow the air out from the first air outlet, and the fan includes:
a volute;
an impeller, rotatably mounted in the volute; and
a motor, mounted in the volute and connected to the impeller, and configured to drive the impeller to rotate;
where the first heat-dissipating member is mounted on the motor and connected to the housing, and the first heat-dissipating member is configured to conduct heat generated by the motor to the housing.

It can be seen from the above technical solutions that in the anesthesia machine proposed in the first aspect of the disclosure, the first heat-dissipating member is provided to conduct the heat generated by the motor to the housing and then dissipate the heat from the housing, so as to cool the motor. This heat dissipation structure is simple and low in cost.

### BRIEF DESCRIPTION OF THE DRAWINGS

In order to more clearly describe the technical solutions in the embodiments of the disclosure, the drawings required for describing the embodiments will be briefly described below. Apparently, the drawings in the following description show some of the embodiments of the disclosure, and those of ordinary skill in the art may still derive other drawings from these drawings without creative efforts.

FIG. 1 is a schematic block diagram of a partial structure of an anesthesia machine according to an embodiment of the disclosure;

FIG. 2 is a connection schematic diagram of a driving gas branch, a fresh gas branch and a breathing circuit of an anesthesia machine according to an embodiment of the disclosure;

FIG. 3 is a schematic structural diagram a fan assembly according to an embodiment of the disclosure;

FIG. 4 is a schematic exploded diagram of a fan assembly according to an embodiment of the disclosure in a first view;

FIG. 5 is a schematic exploded diagram of a fan assembly according to an embodiment of the disclosure in a second view;

FIG. 6 is a schematic structural diagram of a first heat-dissipating member according to an embodiment of the disclosure;

FIG. 7 is a schematic structural diagram of a first elastic supporting member according to an embodiment of the disclosure; and

FIG. 8 is a schematic structural diagram of a second elastic supporting member according to an embodiment of the disclosure.

### DESCRIPTION OF THE PREFERRED EMBODIMENTS

The technical solutions of the embodiments of the disclosure will be described below clearly and comprehensively in conjunction with accompanying drawings of the embodiments of the disclosure. Apparently, the embodiments described are some of, rather than all of, the embodiments of the disclosure. All other embodiments obtained by persons of ordinary skill in the art based on the embodiments of the disclosure without creative efforts shall fall within the scope of protection of the disclosure.

It should also be understood that the terms used in the description of the disclosure are only intended to describe specific embodiments, but not to limit the disclosure. As used in the description and the appended claims of the disclosure, unless the context clearly indicates otherwise, the singular forms “a”, “an” and “the” are intended to include the plural forms.

It should also be further understood that the term “and/or” used in the description and the appended claims of the disclosure refers to one of the items listed correlatively or any combination and all possible combinations of the items, and includes these combinations.

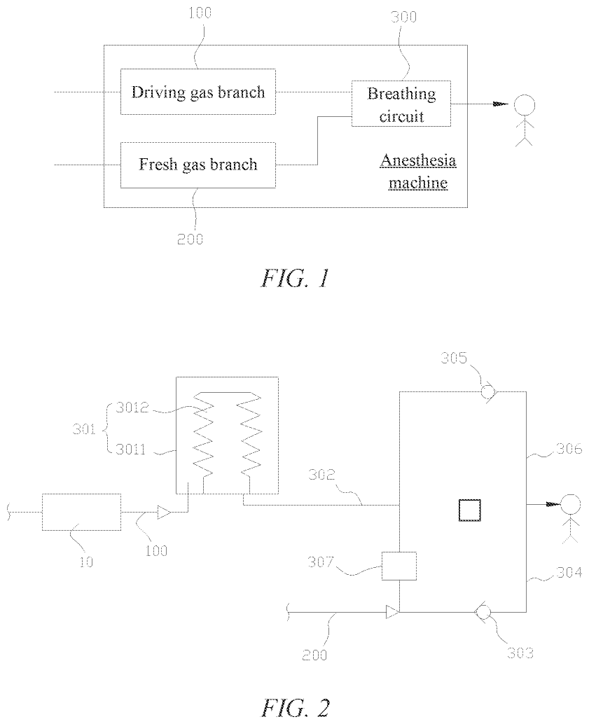

As shown in FIG. 1 to FIG. 5, an embodiment of the disclosure provides an anesthesia machine, including a driving gas branch 100, a fresh gas branch 200, and a breathing circuit 300. The fresh gas branch 200 is configured to deliver fresh gas with anesthetic gas into the breathing circuit 300. The driving gas branch 100 is configured to push the fresh gas from the breathing circuit 300 to a patient. The driving gas branch 100 includes a fan assembly 10. The fan assembly 10 includes a housing 11 and a fan 12. The housing 11 includes a first inner cavity 111, a first air inlet 112 communicating with the first inner cavity 111, and a first air outlet 113 communicating with the first inner cavity 111. The fan 12 is disposed in the first inner cavity 111. The fan 12 is configured to drive air from the first air inlet 112 into the first inner cavity 111 and blow the air out from the first air outlet 113. The fan 12 includes a volute 121, an impeller (not shown in the figures) and a motor 123, the impeller is rotatably mounted in the volute 121, and the motor 123 is mounted in the volute 121 and connected to the impeller, and is configured to drive the impeller to rotate. The first air inlet 112 is located close to the motor 123 so that most of air entering the first inner cavity 111 from the first air inlet 112 flows over a surface of the motor 123 to carry away heat generated in operation of the motor 123.

Optionally, the first air inlet 112 is opposite to the motor 123, and the opposite arrangement not only includes the first air inlet 112 and the motor 123 being directly opposite each other in an air intake direction of the first air inlet 112, but also includes the first air inlet 112 and the motor 123 partially overlapping in the air intake direction of the first air inlet 112, provided that an airflow entering the first inner cavity 111 from the first air inlet 112 can be blown to the motor 123.

By way of example, FIG. 2 shows a connection relationship of the driving gas branch 100, the fresh gas branch 200 and the breathing circuit 300 in an embodiment. The breathing circuit 300 includes a mechanically controlled driving assembly 301, a main pipeline 302, an inhalation check valve 303, a gas supply pipeline 304, an exhalation check valve 305, a gas return pipeline 306 and a carbon dioxide absorption tank 307. The gas supply pipeline 304 is connected to one end of the main pipeline 302, and the inhalation check valve 303 and the carbon dioxide absorption tank 307 are mounted in the gas supply pipeline 304. The gas return pipeline 306 connects the gas supply pipeline 304 to the main pipeline 302, and the exhalation check valve 305 is mounted in the gas return pipeline 306. The mechanically controlled driving assembly 301 includes an air bellow 3011 and a folding airbag 3012. The folding airbag 3012 communicates with the other end of the main pipeline 302. The driving gas branch 100 communicates with a cavity enclosed by the air bellow 3011 and the folding airbag 3012. The fresh gas branch 200 is connected between the inhalation check valve 303 and the carbon dioxide absorption tank 307. During operation, gas that a patient exhales enters the folding airbag 3012 through the exhalation check valve 305 and the main pipeline 302, the driving gas branch 100 provides a driving gas into the cavity enclosed by the air bellow 3011 and the folding airbag 3012, and the driving gas compresses the folding airbag 3012, so that the gas that the patient exhales is delivered to the carbon dioxide absorption tank 307 through the main pipeline 302, the carbon dioxide absorption tank 307 absorbs carbon dioxide in the exhaled gas, and gas discharged from the carbon dioxide absorption tank 307 is mixed with the fresh gas supplied by the fresh gas branch 200 and then delivered to the patient through the gas supply pipeline 304.

In the anesthesia machine proposed in this embodiment, since the first air inlet 112 is disposed opposite the motor 123, the airflow entering the first inner cavity 111 from the first air inlet 112 to the motor 123 can carry away the heat generated in operation of the motor 123 so as to cool the motor 123. This embodiment ingeniously utilizes the structural design of the fan assembly 10 itself, and this heat dissipation structure is simple and low in cost.

Optionally, the volute 121 includes a second inner cavity 1211, a second air inlet 1212 communicating with the second inner cavity 1211, and a second air outlet 1213 communicating with the second inner cavity 1211. The impeller is disposed in the second inner cavity 1211, the second air outlet 1213 communicates with the first air outlet 113, and the second air inlet 1212 faces away from the first air inlet 112.

Since the second air inlet 1212 faces away from the first air inlet 112, air entering the first inner cavity 111 from the first air inlet 112 will pass through an outer side wall of the volute 121 after passing through the motor 123 and before entering the second air inlet 1212, thus carrying away the heat transferred from the motor 123 to the volute 121 and further cooling the motor 123. Of course, the second air inlet 1212 is not limited to being disposed on the side facing away from the first air inlet 112, but can be disposed in other positions of the volute 121, which depends on the actual design needs.

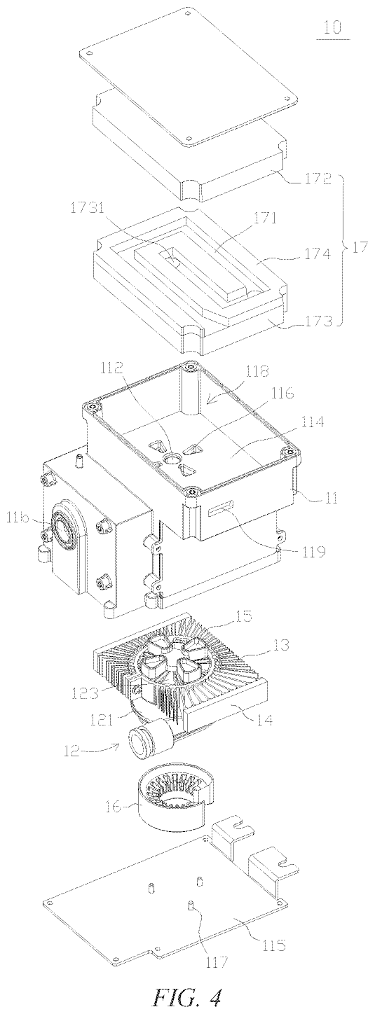

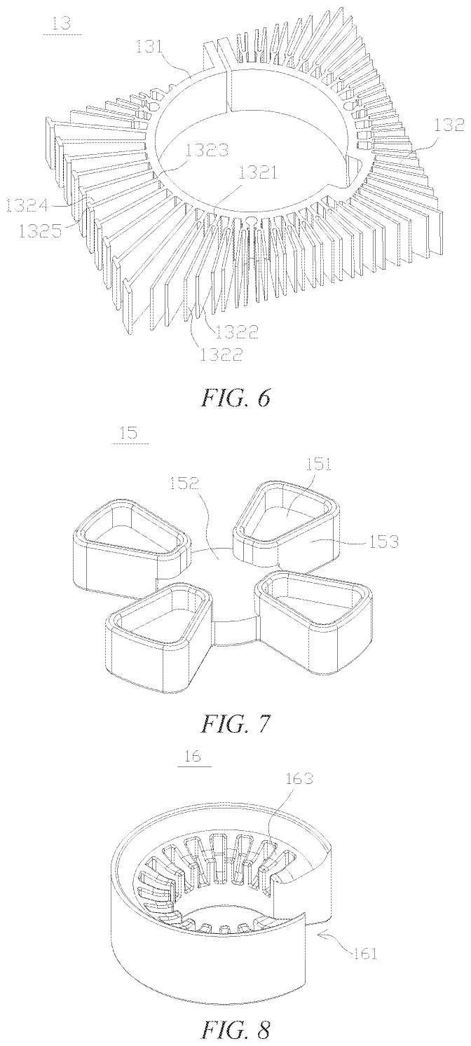

Optionally, as shown in FIG. 4 and FIG. 6, the fan assembly 10 further includes a first heat-dissipating member 13 mounted on the motor 123. The first heat-dissipating member 13 may have an effect of accelerating the heat dissipation of the motor 123 and is simple in structure and low in cost.

Optionally, the first heat-dissipating member 13 includes a connecting portion 131 and radiating fins 132, the connecting portion 131 is connected to the motor 123, and the radiating fins 132 are connected to the connecting portion 131 to expand a heat dissipation area. Of course, the first heat-dissipating member 13 may also be not provided with the radiating fins 132.

Optionally, the connecting portion 131 and the radiating fins 132 are formed integrally, for example, by casting.

Optionally, the first heat-dissipating member 13 is made of a copper-based or aluminum-based metal material, which has good thermal conductivity and can cool the motor 123 well at a low cost.

Optionally, the connecting portion 131 is in the form of a ring, the connecting portion 131 is sleeved over the motor 123, a plurality of radiating fins 132 are provided, and the plurality of radiating fins 132 are spaced around an outer side wall of the connecting portion 131.

Since the connecting portion 131 is sleeved over the motor 123, the motor 123 can be evenly cooled with a good heat dissipation effect. Of course, the connecting portion 131 is not limited to a ring shape, but can also be in other shapes, as long as the connecting portion 131 connects the motor 123 and the radiating fins 132 to cool the motor 123.

Optionally, at least one radiating fin 132 includes a fixed portion 1321 and two radiating sub-fins 1322. One end of the fixed portion 1321 is connected to the connecting portion 131 and the other end of the fixed portion 1321 is connected to the two radiating sub-fins 1322. This embodiment can further achieve the effect of expanding the heat dissipation area and accelerating the heat dissipation of the motor 123.

Optionally, the fan assembly 10 further includes a second heat-dissipating member 14, the second heat-dissipating member 14 is arranged between the first heat-dissipating member 13 and the housing 11, and the second heat-dissipating member 14 is configured to conduct the heat from the first heat-dissipating member 13 to the housing 11 and dissipate the heat from the housing 11.

Optionally, the second heat-dissipating member 14 may be made of a flexible material, such as one of a thermally conductive paste, a thermally conductive adhesive, a thermally conductive pad and a liquid metal. Preferably, the second heat-dissipating member 14 is made of a flexible material, for example, the second heat-dissipating member 14 is a flexible thermally conductive pad, the flexible thermally conductive pad can weaken the vibration of the motor 123 transmitted to the housing 11 via the first heat-dissipating member 13, thus reducing the generation of noise. Further, the first heat-dissipating member 13 may also be made of a flexible material, which can effectively prevent the vibration of the motor 123 from being transmitted to the housing 11.

Optionally, the housing 11 is made of a metal material. The metal material has good thermal conductivity and can quickly conduct the heat generated by the motor 123 to the housing 11 and dissipate the heat from the housing 11. Available metal materials include a copper-based or aluminum-based metal material. Of course, the housing 11 is not limited to being made of a metal material, for example, the housing 11 may also be made of thermally conductive plastic. Available thermally conductive plastics include PP (polypropylene), ABS (Acrylonitrile Butadiene Styrene plastic), PC (Polycarbonate), PA (Nylon Polyamide), LCP (Liquid Crystal Polymer), PPS (Phenylenesulfide), and PEEK (Peek materials).

It should be noted that it is not necessary that the entire housing 11 is made of a metal material or thermally conductive plastic. In some embodiments, only part of the first heat-dissipating member 13 is in contact with the housing 11 via the second heat-dissipating member 14. In this embodiment, only the side wall of the housing 11 that is in contact with the second heat-dissipating member 14 is made of a metal material or thermally conductive plastic.

Optionally, the radiating fins 132 each include a first end 1323 and a second end 1324, the first end 1323 is connected to the connecting portion 131, the second ends 1324 of at least some of the radiating fins 132 are provided with contact portions 1325 at included angles to the radiating fins 132, a plurality of contact portions 1325 are arranged in the same plane, or a plurality of contact portions 1325 are connected to form a contact plate, and the second heat-radiating member 14 is sandwiched between the contact portions 1325 and the housing 11.

The contact portions 1325 are provided, and the plurality of contact portions 1325 are arranged in the same plane or connected to form the contact plate, so the connection between the radiating fins 132 and the housing 11 is facilitated, and the contact area between the radiating fins 132 and the housing 11 can also be expanded, thus accelerating the conduction of heat from the radiating fins 132 to the housing 11.

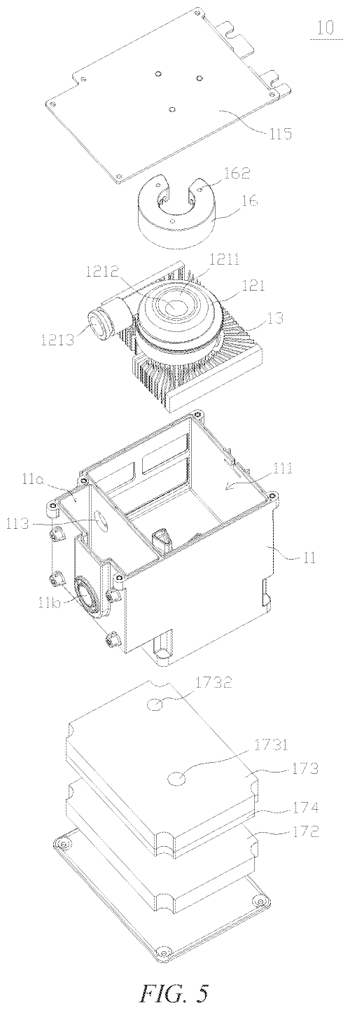

As shown in FIG. 4, FIG. 5, FIG. 7 and FIG. 8, optionally, the fan assembly 10 further includes a first elastic supporting member 15 and a second elastic supporting member 16, the housing 11 includes a top plate 114 and a bottom plate 115 opposite the top plate 114, the first elastic supporting member 15 is sandwiched between the top plate 114 and the top of the fan 123, and the second elastic supporting member 16 is sandwiched between the bottom plate 115 and the bottom of the fan 123. The first elastic supporting member 15 and the second elastic supporting member 16 may function in damping to prevent the vibration of the fan 123 during operation from being transmitted directly to the housing 11 to result in noise.

Optionally, the first elastic supporting member 15 and the second elastic supporting member 16 are made of a silicone material.

Optionally, the top plate 114 of the housing 11 is provided with a mounting portion 116 protruding toward the first inner cavity 111, the first elastic supporting member 15 is provided with a mounting recess 151 matching the mounting portion 116 in shape, the first elastic supporting member 15 is mounted to the top plate 114 by embedding the mounting portion 116 into the mounting recess 151, and the top of the fan 123 abuts against the first elastic supporting member 15.

Exemplarily, four mounting portions 116 are provided, and the four mounting portions are equally spaced in a circular pattern. The first elastic supporting member 15 includes a body 152 and four parts 153 equally spaced around the body 152, each part 153 is provided with a mounting recess 151, and the four mounting portions 116 are embedded in the four parts 153, respectively.

Optionally, the second elastic supporting member 16 is disposed around the second air inlet 1212, and a side wall of the second elastic supporting member 16 is provided with a gap 161 communicating with the second air inlet 1212. Gas enters the second air inlet 1212 through the gap 161 and enters the second inner cavity 1211 through the second air inlet 1212. In some other embodiments, the side wall of the second elastic supporting member 16 may not be provided with the gap 161, for example, the second elastic supporting member 16 may be disposed apart from the bottom plate 115, and the gas enters the second air inlet 1212 from the side of the second elastic supporting member 16 facing the bottom plate 115.

Optionally, the bottom plate 115 of the housing 11 is provided with a mounting post 117 in a protruding manner, the second elastic supporting member 16 is provided with a mounting hole 162 at the bottom, the second elastic supporting member 16 is mounted to the bottom plate 115 by embedding the mounting post 117 into the mounting hole 162, and the bottom of the fan 123 abuts against the second elastic supporting member 16. Exemplarily, three mounting posts 117 are provided, three mounting holes 162 are provided, and the three mounting posts 117 are embedded into the three mounting holes 162, respectively.

Optionally, an inner side wall of the second elastic supporting member 16 is provided with a plurality of supporting ribs 163 spaced apart from each other, the bottom of the fan 123 abuts against the supporting ribs 163, and the supporting ribs 163 can reduce the contact area between the fan 123 and the second elastic supporting member 16, thus reducing the vibration of the fan 123 transmitted to the bottom plate 115 through the second elastic supporting member 16.

Optionally, the anesthesia machine further includes an air fan (not shown in the figures) disposed outside the housing 11. The air fan is configured to generate an airflow blowing toward the housing 11, and the airflow may carry away the heat from the housing 11 and accelerate the heat dissipation of the housing 11, thereby accelerating the heat dissipation of the motor 123.

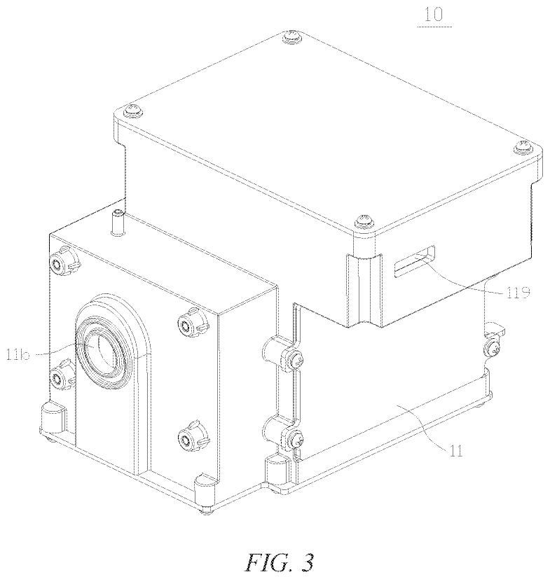

Optionally, as shown in FIG. 3 to FIG. 5, the housing 11 further includes a third inner cavity 118 and a third air inlet 119 communicating with the third inner cavity 118, and the third inner cavity 118 communicates with the first inner cavity 111 via the first air inlet 112. The fan assembly 10 further includes a first noise reduction assembly 17 disposed in the third inner cavity 118, the first noise reduction assembly 17 being configured to reduce noise of the fan from the third air inlet 119.

Since the first noise reduction assembly 17 is disposed in the third inner cavity 118 to reduce noise of the fan from the third air inlet 119, the impact of noise on the patient can be effectively reduced.

Optionally, the first noise reduction assembly 17 is provided with a noise reduction channel 171, one end of the noise reduction channel 171 communicating with the first air inlet 112 and the other end of the noise reduction channel 171 communicating with the third air inlet 119. On the one hand, the noise reduction channel 171 is configured to connect the first air inlet 112 to the third air inlet 119 so that air outside the fan assembly 10 can enter the first inner cavity 111 through the third air inlet 119, the noise reduction channel 171 and the first air inlet 112. On the other hand, the noise reduction channel 171 is configured to reduce the noise generated in operation of the fan 123 from spreading out of the third air inlet 119.

Optionally, the noise reduction channel 171 is spiral. The noise in the spiral noise reduction channel 171 can be well absorbed by the first noise reduction assembly 17 after multiple turns, thereby achieving a better attenuation effect on the noise. Of course, the noise reduction channel 171 is not limited to be spiral. For example, the noise reduction channel 171 may also be serpentine, L-shaped, linear, or honeycomb-shaped, which depends on the actual design needs.

Optionally, the first noise reduction assembly 17 includes an upper clamp plate 172, a lower clamp plate 173, and a spiral structural member 174 sandwiched between the upper clamp plate 172 and the lower clamp plate 173. The upper clamp plate 172, the lower clamp plate 173, and the spiral structural member 174 enclose to form the noise reduction channel 171, and the lower clamp plate 173 is provided with an opening 1731 connecting the noise reduction channel 171 to the first air inlet 112. The first noise reduction assembly 17 composed of the upper clamp plate 172, the lower clamp plate 173 and the spiral structural member 174 is easy to manufacture and assemble.

Optionally, the first noise reduction assembly 17 is made of a porous foam material. Exemplarily, the porous foam material may be acoustic foam, which has a good sound absorption effect and is low in cost.

Optionally, one of the first noise reduction assembly 17 and the top plate 114 is provided with a mistake-proofing hole 1732, and the other of the first noise reduction assembly 17 and the top plate 114 is provided with a mistake-proofing post (not shown in the figures) that penetrates the mistake-proofing hole 1732. The matching between the mistake-proofing post and the mistake-proofing hole 1732 facilitates assembling of the first noise reduction assembly 17, which can effectively prevent the first noise reduction assembly 17 from blocking the first air inlet 112 and the third air inlet 119 due to incorrect mounting of the first noise reduction assembly 17.

Optionally, the housing 11 further includes a fourth inner cavity 11a and a third air outlet 11b communicating with the fourth inner cavity 11a, and the fourth inner cavity 11a communicates with the first inner cavity 111 via the first air outlet 113.

Optionally, a second noise reduction assembly is disposed in the fourth inner cavity 11a. The second noise reduction assembly is configured to reduce noise of the fan from the third air outlet 11b. The second noise reduction assembly can be arranged in the same way as the first noise reduction assembly 17, which will not be described herein again.

Optionally, an inner side wall of the first inner cavity 111 is provided with noise reduction sponge to further reduce the noise in operation of the fan 123.

As shown in FIG. 1 to FIG. 5, an embodiment of the disclosure further provides a veterinary anesthesia machine, including a driving gas branch 100, a fresh gas branch 200, and a breathing circuit 300. The fresh gas branch 200 is configured to deliver fresh gas with anesthetic gas into the breathing circuit 300. The driving gas branch 100 is configured to push the fresh gas from the breathing circuit 300 to a patient. The driving gas branch 100 includes a fan assembly 10. The fan assembly 10 includes a housing 11, a fan 12, and a first heat-dissipating member 13. The housing 11 includes a first inner cavity 111, a first air inlet 112 communicating with the first inner cavity 111, and a first air outlet 113 communicating with the first inner cavity 111. The fan 12 is disposed in the first inner cavity 111. The fan 12 is configured to drive air from the first air inlet 112 into the first inner cavity 111 and blow the air out from the first air outlet 113. The fan 12 includes a volute 121, an impeller (not shown in the figures) and a motor 123, the impeller is rotatably mounted in the volute 121, and the motor 123 is mounted in the volute 121 and connected to the impeller, and is configured to drive the impeller to rotate. The first heat-dissipating member 13 is mounted to the motor 123 and connected to the housing 11, and the first heat-dissipating member 13 is configured to conduct heat generated by the motor 123 to the housing 11.

The above descriptions are merely the specific embodiments of the disclosure, but the scope of protection of the disclosure is not limited thereto, those skilled in the art would readily think of various equivalent modifications or substitutions within the technical scope disclosed in the disclosure, and these modifications or substitutions should all be intended to be included within the scope of protection of the disclosure. Therefore, the scope of protection of the disclosure shall be subject to the scope of protection of the claims.

What is claimed is:

1. An anesthesia machine, comprising a driving gas branch, a fresh gas branch, and a breathing circuit, wherein the fresh gas branch is configured to deliver fresh gas with anesthetic gas into the breathing circuit, the driving gas branch is configured to push the fresh gas from the breathing circuit to a patient, the driving gas branch comprises a fan assembly, the fan assembly comprises a housing, a fan, and a first heat-dissipating member, the housing comprises a first inner cavity, a first air inlet communicating with the first inner cavity, and a first air outlet communicating with the first inner cavity, the fan is disposed in the first inner cavity and configured to drive air from the first air inlet into the first inner cavity and blow the air out from the first air outlet, and the fan comprises:
a volute;
an impeller, rotatably mounted in the volute; and
a motor, mounted in the volute and connected to the impeller, and configured to drive the impeller to rotate;
wherein the first heat-dissipating member is mounted on the motor and connected to the housing, and the first heat-dissipating member is configured to conduct heat generated by the motor to the housing.

2. The anesthesia machine of claim 1, wherein the first heat-dissipating member comprises:
a connecting portion connected to the motor; and
radiating fins connected to the connecting portion to expand a heat dissipation area.

3. The anesthesia machine of claim 2, wherein the connecting portion is in the form of a ring, the connecting portion is sleeved over the motor, a plurality of radiating fins are provided, and the plurality of radiating fins are spaced around an outer side wall of the connecting portion.

4. The anesthesia machine of claim 3, wherein the fan assembly further comprises a second heat-dissipating member, the second heat-dissipating member is arranged between the first heat-dissipating member and the housing, and the second heat-dissipating member is configured to direct the heat from the first heat-dissipating member to the housing.

5. The anesthesia machine of claim 4, wherein the radiating fins each comprise a first end and a second end, the first end is connected to the connecting portion, the second ends of at least some of the radiating fins are provided with contact portions at included angles to the radiating fins, a plurality of contact portions are arranged in the same plane, or a plurality of contact portions are connected to form a contact plate, and the second heat-radiating member is sandwiched between the contact portions and the housing.

6. The anesthesia machine of claim 4, wherein
the housing is made of a metal material; or
the housing is made of thermally conductive plastic; or
a side wall of the housing that is in contact with the second heat-dissipating member is made of a metal material or thermally conductive plastic.

7. The anesthesia machine of claim 1, wherein the first air inlet is located close to the motor so that most of gas entering the first inner cavity from the first air inlet flows over a surface of the motor to carry away the heat generated in operation of the motor.

8. The anesthesia machine of claim 1, wherein the volute comprises a second inner cavity, a second air inlet communicating with the second inner cavity, and a second air outlet communicating with the second inner cavity, the impeller is disposed in the second inner cavity, the second air outlet communicates with the first air outlet, and the second air inlet is provided close to the first air inlet.

9. The anesthesia machine of claim 8, wherein the fan assembly further comprises a first elastic supporting member and a second elastic supporting member, the housing comprises a top plate and a bottom plate opposite the top plate, the first elastic supporting member is sandwiched between the top plate and the top of the fan, and the second elastic supporting member is sandwiched between the bottom plate and the bottom of the fan.

10. The anesthesia machine of claim 9, wherein the second elastic supporting member is disposed around the second air inlet, and a side wall of the second elastic supporting member is provided with a gap communicating with the second air inlet.

11. The anesthesia machine of claim 1, wherein the anesthesia machine further comprises an air fan disposed outside the housing, the air fan being configured to generate an airflow to the housing.

12. The anesthesia machine of claim 4, wherein the first heat-dissipating member is connected to the housing by the second heat-dissipating member, and at least one of the first heat-dissipating member and the second heat-dissipating member is made of a flexible material.

13. An anesthesia machine, wherein the anesthesia machine comprises a driving gas branch, a fresh gas branch, and a breathing circuit, wherein the fresh gas branch is configured to deliver fresh gas with anesthetic gas into the breathing circuit, the driving gas branch is configured to push the fresh gas from the breathing circuit to a patient, the driving gas branch comprises a fan assembly, and the fan assembly comprises:
a housing, comprising a first inner cavity, a first air inlet communicating with the first inner cavity, a first air outlet communicating with the first inner cavity, a third inner cavity, and a third air inlet communicating with the third inner cavity, the third inner cavity communicating with the first inner cavity via the first air inlet;
a fan, disposed in the first chamber and configured to drive air from the third air inlet into the first inner cavity and blow the air out from the first air outlet; and
a first noise reduction assembly, disposed in the third inner cavity and configured to reduce noise.

14. The anesthesia machine of claim 13, wherein the first noise reduction assembly is provided with a noise reduction channel, one end of the noise reduction channel communicating with the first air inlet and the other end of the noise reduction channel communicating with the third air inlet.

15. The anesthesia machine of claim 14, wherein the noise reduction channel is linear, L-shaped, spiral, honeycomb-shaped, or wavy.

16. The anesthesia machine of claim 15, wherein the first noise reduction assembly comprises an upper clamp plate, a lower clamp plate and a spiral structural member sandwiched between the upper clamp plate and the lower clamp plate, wherein the upper clamp plate, the lower clamp plate and the spiral structural member enclose to form the noise reduction channel, and the lower clamp plate is provided with an opening connecting the noise reduction channel to the first air inlet.

17. An anesthesia machine, comprising a driving gas branch, a fresh gas branch, and a breathing circuit, wherein the fresh gas branch is configured to deliver fresh gas with anesthetic gas into the breathing circuit, the driving gas branch is configured to push the fresh gas from the breathing circuit to a patient, the driving gas branch comprises a fan assembly, the fan assembly comprises a housing, a fan, the housing comprises a first inner cavity, a first air inlet communicating with the first inner cavity, and a first air outlet communicating with the first inner cavity, the fan is disposed in the first inner cavity and configured to drive air from the first air inlet into the first inner cavity and blow the air out from the first air outlet, and the fan comprises:
a volute;
an impeller, rotatably mounted in the volute; and
a motor, mounted in the volute and connected to the impeller, and configured to drive the impeller to rotate; the first air inlet is disposed opposite the motor.

18. The anesthesia machine of claim 17, wherein the volute includes a second inner cavity, a second air inlet communicating with the second inner cavity, and a second air outlet communicating with the second inner cavity; the impeller is disposed in the second inner cavity, the second air outlet communicates with the first air outlet, and the second air inlet faces away from the first air inlet.

19. The anesthesia machine of claim 17, wherein the housing further comprises a third inner cavity, and a third air inlet communicating with the third inner cavity, the third inner cavity communicating with the first inner cavity via the first air inlet; a first noise reduction assembly is disposed in the third inner cavity and configured to reduce noise.

20. The anesthesia machine of claim 19, wherein the first noise reduction assembly is provided with a noise reduction channel, one end of the noise reduction channel communicating with the first air inlet and the other end of the noise reduction channel communicating with the third air inlet.
